(19) United States
(12) Patent Application Publication
(10) Pub. No.: US 2011/0027293 A1
(43) Pub. Date: Feb. 3, 2011

(54) METHODS AND COMPOSITIONS FOR THE INHIBITION OF VIRAL INFECTION BY TARGETING INTRACELLULAR TRAFFICKING

(76) Inventor: Ali Munawar, Dedham, MA (US)

Correspondence Address: CLARK & ELBING LLP, 101 FEDERAL STREET, BOSTON, MA, 02110, US

(21) Appl. No.: 12/644,799

(22) Filed: Dec. 22, 2009

Related U.S. Application Data
(60) Provisional application No. 61/139,777, filed on Dec. 22, 2008.

(51) Int. Cl. A61K 39/42; A61P 31/20; A61K 38/02; A61K 39/395; A61K 31/713; A61K 39/12; C07K 2/00; C07K 16/08

(52) U.S. Cl. 424/159.1; 514/3.7; 424/130.1; 514/44 A; 424/204.1; 530/300; 424/186.1; 530/389.4

(57) ABSTRACT

The present invention features methods and compositions for the treatment or inhibition of infection by human papillomavirus (HPV) in a subject by administering an agent (e.g., a small molecule or antibody) that binds the carboxy-terminal region of the HPV L2 protein and reduces or inhibits the binding of the L2 protein to an intracellular molecular motor protein (IMMP) or IMMP co-factor. The invention also features a method for treating or inhibiting an HPV infection by administering a nucleic acid molecule that, for example, decreases the expression of the HPV L2 protein.

### CROSS-REFERENCE TO RELATED APPLICATIONS

This application claims the benefit of the filing date of U.S. provisional application 61/139,777, filed Dec. 22, 2008, herein incorporated by reference.

### BACKGROUND OF THE INVENTION

Viruses are obligate cellular parasites that have evolved the ability to exploit the molecular machinery of an infected host cell. Infection by viruses that replicate within the nucleus requires the passage of the virus particle through the cytoplasm of the infected cell, which creates a high diffusion barrier due to its viscosity and the presence of a dense network of cytoskeleton proteins. Viruses, including human papillomavirus (HPV), have acquired the ability to utilize the intracellular transport system of an infected mammalian cell to travel through the cytoplasm. Such viruses bind to molecular motor proteins (e.g., kinesin, dynein, and myosin), which are responsible for delivering the viral particle to the microtubule organization center, nucleus, or other sites within the cell during viral infection. There exists a need in the art for anti-viral therapies that target the interaction between viral proteins and intracellular molecular motor proteins (IMMPs) and IMMP co-factors.

### SUMMARY OF THE INVENTION

A first aspect of the present invention features methods and compositions for the treatment or inhibition of infection by human papillomavirus (HPV) infection in a subject by administering an agent (e.g., a small molecule or antibody) that binds the carboxy-terminal region of the HPV L2 protein and reduces or inhibits the binding of the L2 protein to an intracellular molecular motor protein (IMMP) or IMMP co-factor. The invention also features a method for treating or inhibiting an HPV infection by administering a nucleic acid molecule that, for example, decreases the expression of the HPV L2 protein.

In one embodiment, the invention features a method of inhibiting or treating HPV infection in a subject by administering a therapeutic agent that binds a carboxy-terminal region of the HPV L2 protein, thereby reducing or inhibiting the binding of the L2 protein to an IMMP or IMMP co-factor and, thus, inhibiting or treating the HPV infection. The HPV L2 protein may include all or a portion of the amino acid sequences of SEQ ID NOs:1-10. The carboxy-terminal region of the L2 protein may include, for example, the last 49 to 200 amino acid residues of the L2 protein and may include the amino acid sequence PSLIPIVPGSPQYTIIADGGDFYLHPSYYMLRKRRKRLPYFFS DVSLAA. The therapeutic agent that binds to the carboxy-terminal region of the HPV L2 protein may be a small molecule, an antibody (e.g., an antibody that binds a linear or conformational epitope within the carboxy-terminal region), or a peptidomimetic. The therapeutic agent may be administered to a subject (e.g., a human), e.g., by injection and may be administered alone or in combination with one or more additional therapeutic agents, such as an anti-viral agent, an immunostimulatory agent, or an immunization vaccine.

A second aspect of the invention also features a method of inhibiting or treating HPV infection in a subject by administering a nucleic acid molecule that reduces or inhibits expression of L2 protein in a cell infected with HPV, thereby reducing the amount of L2 protein available for binding to an IMMP or IMMP co-factor and, thus, inhibiting or treating an HPV infection. The nucleic acid molecule may be an antisense nucleic acid, a peptide nucleic acid, RNAi, shRNAi, siRNA, or micro RNAi. The nucleic acids may decrease the expression of L2 protein in a cell by at least 50% (e.g., 50, 55, 60, 65, 70, 75, 80, 85, 90, 95, 99, or 100%) relative to a cell not exposed to the nucleic acid molecule.

A third aspect of the invention features a pharmaceutical composition that includes an anti-L2 agent capable of binding the carboxy-terminal region of HPV L2 protein and reducing or inhibiting an interaction between the L2 protein and an IMMP or IMMP co-factor. The anti-L2 agent may be a small molecule, peptide, or antibody. In several embodiments, the antibody, peptide, or small molecule may, for example, specifically bind to the L2 protein with a dissociation constant of less than 10⁻⁷M (e.g., 10⁻⁷, 10⁻⁸, 10⁻⁹, 10⁻¹⁰, 10⁻¹¹, 10⁻¹², 10⁻¹³, 10⁻¹⁴, or 10⁻¹⁵ M.) The antibody binds all or a portion of the HPV L2 protein, e.g., all or a portion of the amino acid sequence of one or more of SEQ ID NOs:1-10. The composition of the invention may further include a pharmaceutically acceptable diluent, excipient, carrier, or adjuvant. The composition may alleviate one or more symptoms associated with HPV infection when administered to a subject (e.g., a human).

In a fourth aspect, the invention features a pharmaceutical composition that includes a peptide having all or a portion of the carboxy-terminal region of the HPV L2 protein. The peptide may be capable of eliciting an immune response that protects a subject (e.g., a human) against HPV infection. The HPV L2 protein bound may include all or a portion of one or more of the amino acid sequences of SEQ ID NOs:1-10. The composition may be formulated as a vaccine that inhibits HPV infection. The composition may be administered prior to exposure to HPV or, alternatively, the composition may be administered following exposure to HPV. Upon administration, the composition may alleviate one or more symptoms associated with HPV.

In an embodiment of all aspects of the present invention, the IMMP may be, without limitation, dynein, myosin, or kinesin and the IMMP co-factor may be dynactin or dynein light chain (DLC). The strain of HPV infecting the subject may be HPV 1, 2, 4, 6, 7, 10, 11, 16, 18, 31, 32, 33, 35, 39, 42, 43, 44, 45, 51, 55, 56, 58, 59, or 68, or any other known strain of HPV.

As used herein, by “administering” is meant a method of giving a dosage of a pharmaceutical composition to a subject. The compositions utilized in the methods described herein can be administered by a route selected from, e.g., parenteral, dermal, transdermal, ocular, inhalation, buccal, sublingual, perilingual, nasal, rectal, topical, and oral. Parenteral administration includes intra-arterial, intravenous, intraperitoneal, subcutaneous, and intramuscular administration. The preferred method of administration can vary depending on various factors (e.g., the components of the composition being administered and the severity of the condition being treated).

By “an amount sufficient to treat” is meant the amount of a composition administered to improve, inhibit, or ameliorate a condition of a subject, or a symptom of a viral infection (e.g., an HPV infection), in a clinically relevant manner (e.g., improve, inhibit, or ameliorate infection by a virus (e.g., HPV) or one or more symptoms that occur following infection (e.g., HPV infection)). Any improvement resulting from compositions of the invention or the use of a method of the invention in the subject is considered sufficient to achieve treatment. Preferably, an amount sufficient to treat is an amount that reduces, inhibits, or prevents the occurrence or one or more symptoms of a viral (e.g., HPV) infection or is an amount that reduces the severity of, or the length of time during which a subject suffers from, one or more symptoms of the infection (e.g., by at least 10%, 20%, or 30%, more preferably by at least 50%, 60%, or 70%, and most preferably by at least 80%, 90%, 95%, 99%, or more, relative to a control subject that is not treated with a composition or by a method of the invention). A sufficient amount of the pharmaceutical composition of the invention used to practice the methods described herein (e.g., the treatment of viral infection) varies depending upon the manner of administration and the age, body weight, and general health of the subject being treated. A physician or researcher can decide the appropriate amount and dosage regimen.

By “expression” is meant the amount of a gene product or polypeptide produced by transcription or translation, respectively. The amount of expression of a gene product or polypeptide can be detected by methods known to one of skill in the art. For example, polypeptide expression is often detected by Western blotting, gene product expression is often detected by Southern blotting or polymerase chain reaction (PCR), and RNA gene product expression is often detected by Northern blotting, PCR, or RNAse protection assays.

By “HPV-associated disease” is meant any disease or medical condition caused by or associated with an infection of a human papillomavirus. Exemplary HPV-associated diseases include HPV-associated infections, atypical squamous cells of undetermined significance (ASCUS), warts (e.g., anogenital warts, bowenoid papulosis, giant condylomata, cutaneous warts, common warts, plantar warts, flat warts, butcher warts, and epidermodysplasia verruciformis), respiratory papillomatosis, laryngeal papilloma, maxiallary sinus papilloma, conjunctival papillomatosis, oral focal hyperplasia, intraepithelial neoplasia (e.g., cervical intraepithelial neoplasia (CIN), vulval intraepithelial neoplasia (VIN), and anal intraepithelial neoplasia (AIN)), cervical cancer, vulvar cancer, anal cancer, vaginal cancer, prostate cancer, head and neck cancer, squamous cell carcinoma of the lung, squamous cell carcinoma of the sinuses, squamous cell carcinoma of the esophagus, oral carcinoma, conjunctival carcinoma, and other HPV-mediated cancers.

By “inducing an immune response” is meant eliciting a humoral response (e.g., the production of antibodies) or a cellular response (e.g., the activation of T cells) directed against a virus (e.g., HPV) in a subject to which the pharmaceutical composition (e.g., a vaccine) has been administered.

By “pharmaceutical composition” is meant any composition that contains a therapeutically or biologically active agent (e.g., an antisense nucleic acid molecule that inhibits the expression of the HPV L2 protein or an anti-L2 agent (e.g., a small molecule, peptide, or antibody) that reduces or inhibits HPV L2 binding to an IMMP or IMMP co-factor) that is suitable for administration to a subject and that is capable of inducing an immune response against a virus. For the purposes of this invention, pharmaceutical compositions suitable for delivering a therapeutic or biologically active agent can include, e.g., tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

By “pharmaceutically acceptable diluent, excipient, carrier, or adjuvant” is meant a diluent, excipient, carrier, or adjuvant which is physiologically acceptable to the subject while retaining the therapeutic properties of the pharmaceutical composition with which it is administered. One exemplary pharmaceutically acceptable carrier is physiological saline. Other physiologically acceptable diluents, excipients, carriers, or adjuvants and their formulations are known to one skilled in the art.

By “reduce or inhibit” is meant the ability to cause an overall decrease of, preferably, 20% or greater, more preferably 50% or greater, and most preferably 75%, 80%, 85%, 90%, 95%, or greater. For therapeutic applications, to “reduce or inhibit” refers to the incidence of or the symptoms of the infection being treated or the presence or extent of a viral infection being treated. Symptoms of a HPV infection include, for example, genital warts and other lesions. For diagnostic or monitoring applications, to “reduce or inhibit” refers to a decrease in the level of protein or nucleic acid detected by diagnostic assays.

By “small molecule” is meant any therapeutic agent that can be used to treat or inhibit a viral infection (e.g., an HPV infection). As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol.

By “specifically bind” is meant the preferential association of a binding agent (e.g., an antibody, antibody fragment, receptor, ligand, peptide, or small molecule) to a target molecule (e.g., a cytokine, chemokine, hormone, receptor, or ligand) or to a cell or tissue bearing the target molecule (e.g., a cell surface antigen, such as a receptor or ligand) and not to non-target cells or tissues lacking the target molecule. It is recognized that a certain degree of non-specific interaction may occur between a binding moiety and a non-target molecule (present alone or in combination with a cell or tissue). Nevertheless, specific binding may be distinguished as mediated through specific recognition of the target molecule. Specific binding results in a much stronger association between the binding moiety (e.g., an antibody or small molecule) and, e.g., cells bearing the target molecule (e.g., an antigen) than between the binding moiety and, e.g., cells lacking the target molecule. Specific binding typically results in greater than 2-fold, preferably greater than 5-fold, more preferably greater than 10-fold and most preferably greater than 100-fold increase in amount of bound binding moiety (per unit time) to e.g., a cell or tissue bearing the target molecule as compared to a cell or tissue lacking that target molecule. Binding moieties bind to the target molecule with a dissociation constant of e.g., less than 10⁻⁶M, more preferably less than 10⁻⁷M, 10⁻⁸M, 10⁻⁹ M, 10⁻¹⁰ M, 10⁻¹¹ M, or 10⁻¹²M, and most preferably less than 10⁻¹³M, 10⁻¹⁴M, or 10⁻¹⁵M. Specific binding to a protein under such conditions requires a binding moiety that is selected for its specificity for that particular protein. A variety of assay formats are appropriate for selecting binding moieties (e.g., antibodies and small molecules) capable of specifically binding to a particular target molecule. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity. In addition, real-time PCR and GFP expression assays may be used to assess the ability of a compound (e.g., an antibody or small molecule) to specifically bind to, e.g., the carboxy-terminal region of the HPV L2 protein, thereby inhibiting the binding of the L2 protein to an IMMP or IMMP co-factor.

By “subject” is meant any animal, e.g., a mammal (e.g., a human). A subject to be treated according to the methods described herein (e.g., a subject infected with, or at risk of being infected with a virus (e.g., HPV)) may be one who has been diagnosed by a medical practitioner as having such a condition or risk. Diagnosis may be performed by any suitable means. A subject in whom the development of an infection is being prevented may or may not have received such a diagnosis. One skilled in the art will understand that a subject to be treated according to the present invention may have been subjected to standard tests or may have been identified, without examination, as one at high risk due to the presence of one or more risk factors.

By “substantially identical” is meant a polypeptide or nucleic acid exhibiting at least 60%, 65%, 70%, 75%, 80%, 85%, 90%, 95%, 98%, 99%, or even 100% identity to a reference amino acid or nucleic acid sequence over at least 20, 25, 30, 35, 40, 45, 50, 55, 60, 65, or 70, 80, 90, 100, 150, 200, 250, 300, 350, 400, 450, 460, 470, 480, 490, 500, 1000, or 1500 contiguous residues or bases. Sequence identity is typically measured using a sequence analysis program (e.g., BLAST 2; Tatusova et al., FEMS Microbiol Lett. 174: 247-250, 1999) with the default parameters specified therein. Conservative substitutions typically include substitutions within the following groups: glycine, alanine, valine, isoleucine, leucine; aspartic acid, glutamic acid, asparagine, glutamine; serine, threonine; lysine, arginine; and phenylalanine and tyrosine.

By “treating” is meant administering a pharmaceutical composition for prophylactic and/or therapeutic purposes. Prophylactic treatment may be administered, for example, to a subject who is not yet ill, but who is susceptible to, or otherwise at risk of, a particular disorder, e.g., infection with a virus (e.g., HPV). Therapeutic treatment may be administered, for example, to a subject already suffering from a disorder in order to improve or stabilize the subject's condition (e.g., a subject already infected with at least one virus). Thus, in the claims and embodiments described herein, treating is the administration to a subject either for therapeutic or prophylactic purposes. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique. In some instances, treating can result in the inhibition of viral infection, the treatment of the infection(s), and/or the amelioration of one or more symptoms of the infection(s). Confirmation of treatment can be assessed by detecting an improvement in the treated subject (e.g., the absence of symptoms in the subject), or by the inability to detect the presence of the virus in the treated subject. Methods for detecting viruses include, e.g., growth of the virus in cell culture from a specimen taken from the infected subject, detection of virus-specific IgM antibody in, e.g., the blood, detection of viral antigens by ELISA, detection of virus-encoded DNA or RNA by PCR, or observation of virus particles by electron microscopy, or any other method of detection known to one of skill in the art.

The term “vaccine,” as used herein, is defined as material used to provoke an immune response and confer immunity after administration of the vaccine to a subject.

The term “virus,” as used herein, is defined as any virus that infects mammals and any viral strains thereof.

Other features and advantages of the invention will be apparent from the detailed description and from the claims.

### BRIEF DESCRIPTION OF THE DRAWINGS

FIG. 1 is the amino acid sequence of the HPV 16 L2 protein (SEQ ID NO: 1).

FIG. 2 is the amino acid sequence of the HPV 45 L2 protein (SEQ ID NO: 2).

FIG. 3 is the amino acid sequence of the HPV 2 L2 protein (SEQ ID NO: 3).

FIG. 4 is the amino acid sequence of the HPV 13 L2 protein (SEQ ID NO: 4).

FIG. 5 is the amino acid sequence of the HPV 18 L2 protein (SEQ ID NO: 5).

FIG. 6 is the amino acid sequence of the HPV 56 L2 protein (SEQ ID NO: 6).

FIG. 7 is the amino acid sequence of the HPV 32 L2 protein (SEQ ID NO: 7).

FIG. 8 is the amino acid sequence of the HPV 31 L2 protein (SEQ ID NO: 8).

FIG. 9 is the amino acid sequence of the HPV 33 L2 protein (SEQ ID NO: 9).

FIG. 10 is the amino acid sequence of the HPV 35 L2 protein (SEQ ID NO: 10).

FIG. 11 is a graph showing the results of a real-time PCR assay. Anti-L2 antibody was administered to cells and viral gene expression was measured using real-time PCR. The results demonstrate that the administration of anti-L2 antibody results in the loss of HPV gene expression.

FIG. 12 is a graph showing the results of a real-time PCR assay. Anti-dynein antibody was administered to cells and viral gene expression was measured using real-time PCR. The graph shows expression data for control cells.

FIG. 13 is a bar graph showing the percent of viral gene expression (assessed by monitoring GFP expression) after administering anti-dynein or anti-microtubule antibodies to cells infected with a DNA virus, a retrovirus, and an RNA virus. The administration via microinjection of an anti-dynein antibody disrupts the microtubule network, resulting in decreased viral trafficking and gene expression.

FIG. 14 is a graph showing the percent of viral gene expression (assessed by monitoring GFP expression) following administration of an anti-dynein small organic molecule (EHNA). EHNA was dissolved in DMEM and added to several sets of cells infected with a virus at final concentrations of 0.1 μM, 0.2 μM, 0.5 μM, and 1 μM.

FIGS. 15A-15D are photomicrographs showing that treatment of cells with EHNA inhibits infection by MoMLV (FIG. 15A) and hepatitis C virus (HCV; FIG. 15B) relative to infection by MoMLV and HCV in the absence of treatment with EHNA (FIGS. 15C and 15D, respectively) as a result of the loss of cytoplasmic dynein motor activity on the viral lifecycle in treated cells. The levels of infection are based on the inhibition of GFP expression, which was determined by infection of cells by MoMLV and HCV viral constructs that included cloned GFP. To determine the consequences of dynein inhibition on the viral life cycle, the expression of viral genes was correlated to the expression (or loss of expression) of GFP. Pictured are cells infected with MoMLV and Hepatitis C with and without EHNA-induced dynein inhibition. Brightness is an indication of GFP expression.

### DETAILED DESCRIPTION

The invention described herein features methods and compositions for the treatment or inhibition of infection by human papillomavirus (HPV) in a subject by administering a therapeutic agent (e.g., a small molecule, peptide, or antibody) that binds the carboxy-terminal region of the HPV L2 protein and reduces or inhibits the binding of the L2 protein to an intracellular molecular motor protein (IMMP) or IMMP co-factor. The invention also features a method for treating or inhibiting an HPV infection by administering a nucleic acid molecule that, for example, decreases the expression of the HPV L2 protein.

### Human Papillomavirus (HPV)

Papillomaviruses are a diverse group of DNA-based viruses that infect the skin and mucous membranes of humans and other mammals. The HPV virion infects epithelial tissues through micro-abrasions, whereby the virion associates with cellular receptors (e.g., integrins and laminins), leading to entry of the virions into basal epithelial cells through clathrin-mediated endocytosis or caveolin-mediated endocytosis. The viral genome is transported to the nucleus, resulting in the replication of the viral genome. The late genes, L1 and L2, are transcribed and translated and serve as structural proteins that encapsidate the viral genome.

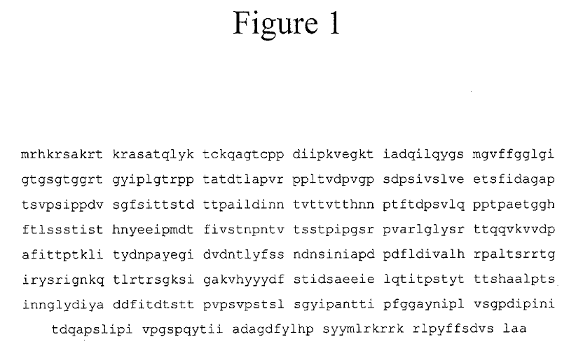

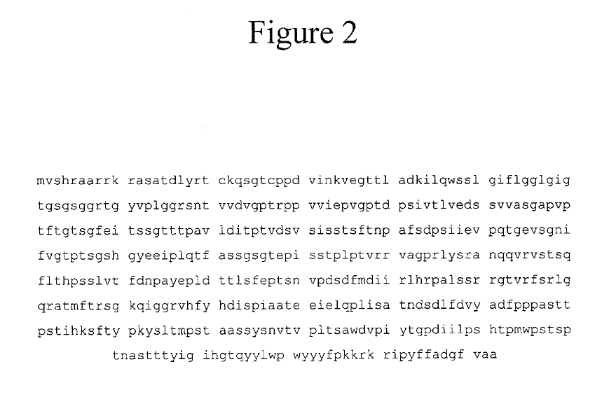

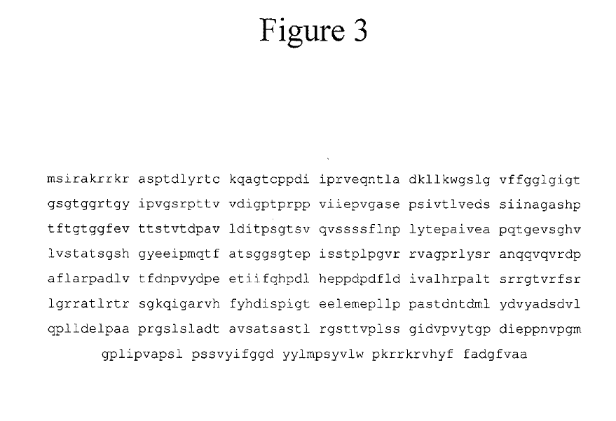

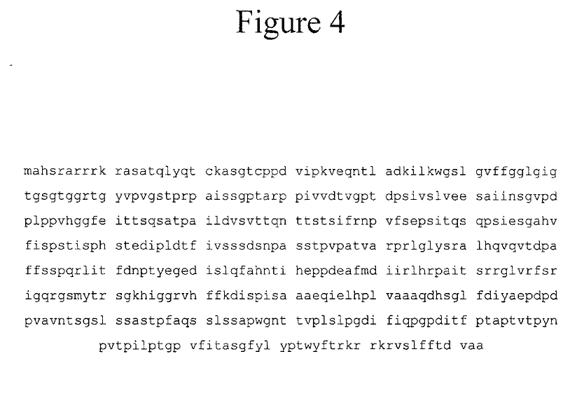

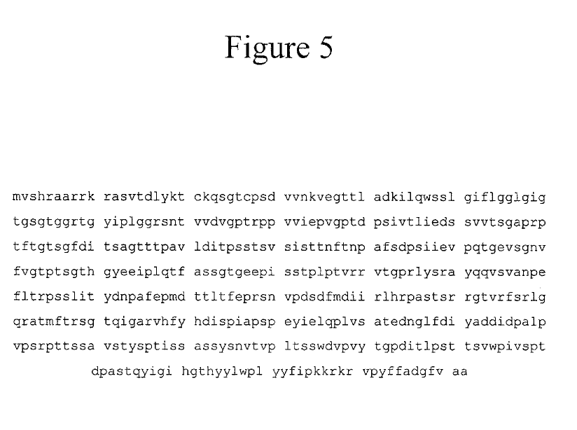

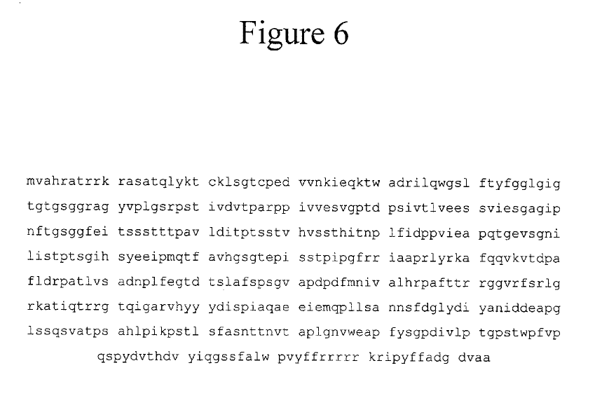

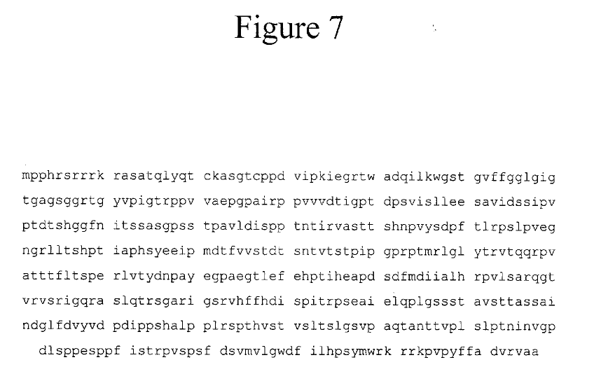

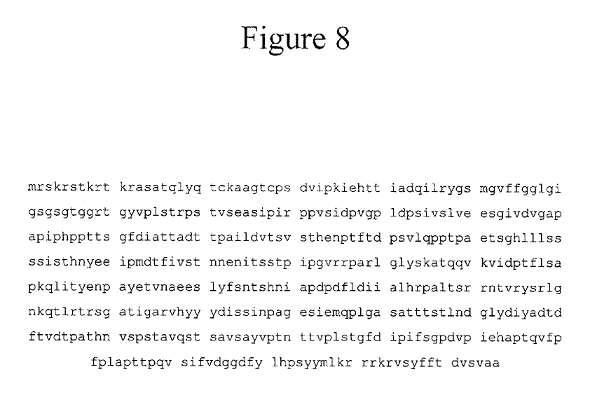

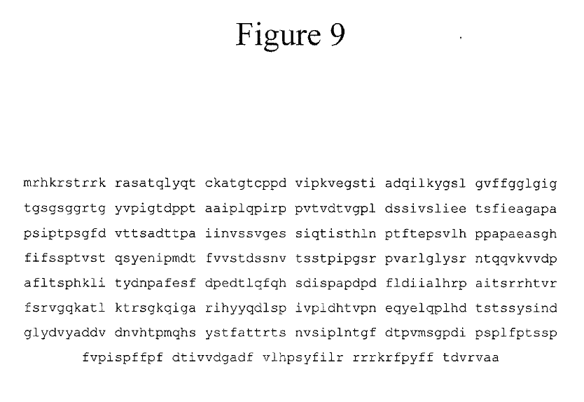

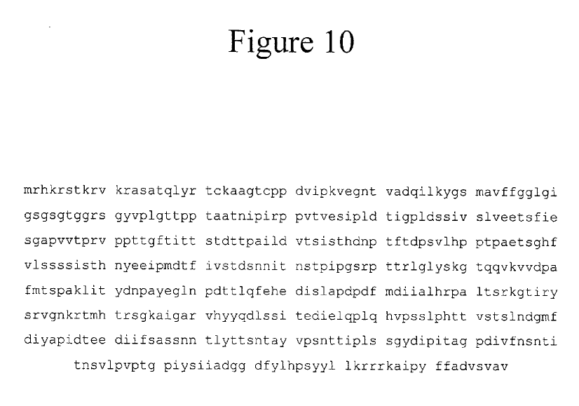

The methods and compositions described herein can be used to treat or prevent HPV infection and HPV-mediated diseases by targeting the interaction between the HPV L2 protein (e.g., the carboxy-terminus of the L2 protein) and IMMPs or IMMP co-factors. The L2 protein is a capsid protein that varies in length based on HPV type. Exemplary L2 amino acid sequences for various HPV subtypes are described in FIGS. 1-10.

The present invention provides therapeutic agents (e.g., small molecules, peptides, or antibodies) that bind to the carboxy-terminus of the HPV L2 protein. The carboxy-terminus of the L2 protein is meant the last 200, 150, 140, 130, 120, 110, 100, 90, 80, 70, 60, 50, 45, 40, 35, 30, 25, 20, 15, or 10 amino acid residues at the carboxy-terminal end of the full-length HPV L2 protein of a given strain or subtype. For example, the carboxy-terminus of HPV 16 (see, e.g., the sequence of FIG. 1) may include amino acid residues in the range of, e.g., 420-473, 421-473, 422-473, 423-473, 424-473, 425-473, 425-472, 425-471, 425-470, or any longer or shorter sequence or any substantially identical sequence. Exemplary carboxy-terminal amino acid sequences for various HPV subtypes are listed in Table 1.

TABLE 1

| Subtype | Exemplary C-Terminal Sequences |
|---|---|
| HPV 2 | PLIPVAPSLPSSVYIFGGDYYLMPSYVLWPKRRKRVHYFFADGFVAA |
| HPV 13 | PILPTGPVFITASGFYLYPTWYFTRKRRKRVSLFFTDVAA |
| HPV 31 | PLAPTTPQVSIFVDGGDFYLHPSYYMLKRRRKRVSYFFTDVSVAA |
| HPV 33 | PISPFFPFDTIVVDGADFVLHPSYFILRRRRKRFPYFFTDVRVAA |
| HPV 35 | PVPTGPIYSIIADGGDFYLHPSYYLLKRRRKAIPYFFADVSVAV |
| HPV 16 | PSLIPIVPGSPQYTIIADGGDFYLHPSYYMLRKRRKRLPYFFSDVSLAA |
| HPV 32 | PVSPSFDSVMVLGWDFILHPSYMWRKRRKPVPYFFADVRVAA |
| HPV 56 | PYDVTHDVYIQGSSFALWPVYFFRRRRRKRIPYFFADGDVAA |
| HPV 18 | PIVSPTPASTQYIGI HGTHYYLWPLYYFIPKKRKRVPYFFADGFVAA |
| HPV 45 | PSTSPTNASTTTYIGIHGTQYYLWPWYYYFPKKRKRIPYFFADGFVAA |

In another embodiment, the therapeutic agent is a nucleic acid molecule (e.g., an antisense nucleic acid, a peptide nucleic acid, RNAi, shRNAi, siRNA, or micro RNAi) that reduces or inhibits the expression of the HPV L2 protein in the infected cell, thereby reducing the amount of HPV L2 available to bind to an IMMP or IMMP co-factor.

Approximately 130 HPV subtypes have been identified. Clinically relevant HPV subtypes include, e.g., HPV 6, 11, 16, 18, 31, 33, 35, 39, 44, 45, 52, 53, 54, 56, 58, 61, 66, 67, 69, CP8304, CP141, MM4, MM7, or MM9, any of which may be treated using the methods and compositions of the present invention. Conditions associated with specific HPV subtypes include, for example, exophytic condyloma (HPV types 6 and 11), flat condyloma, especially of the cervix (HPV types 6, 11, 16, 18, and 31), giant condyloma (HPV types 6 and 11), cervical cancer (HPV types 16, 18, 31, 45, and 33), respiratory and conjunctival papillomatosis (HPV types 6 and 11), and high-grade cervical intraepithelial neoplasia (HPV 31, 33, 35, 39, 44, 45, 52, 53, 54, 56, 58, 61, 66, 67, 69, CP8304, CP141, MM4, MM7, and MM9).

Examples of HPV-mediated diseases include, e.g., HPV-associated infections, atypical squamous cells of undetermined significance (ASCUS), warts (e.g., anogenital warts, bowenoid papulosis, cutaneous warts, common warts, plantar warts, flat warts, butcher warts, and epidermodysplasia verruciformis), laryngeal papilloma, maxiallary sinus papilloma, oral focal hyperplasia, intraepithelial neoplasia (e.g., vulval intraepithelial neoplasia (VIN) and anal intraepithelial neoplasia (AIN)), cervical cancer, vulvar cancer, anal cancer, vaginal cancer, penile cancer, head and neck cancer, squamous cell carcinoma of the lung, squamous cell carcinoma of the sinuses, squamous cell carcinoma of the esophagus, oral carcinoma, conjunctival carcinoma, and other HPV-mediated cancers.

### Intracellular Molecular Motor Proteins (IMMPs) and IMMP Co-Factors

Certain viruses have acquired the ability to utilize the intracellular transport system of an infected cell to travel through the cytoplasm. Such viruses (e.g., hepatitis C, hepatitis B, duck HBV, influenza A, influenza B, respiratory syncytial virus, leukemia virus, HIV-1, adenovirus, herpes simplex virus, Ebola virus, Marburg virus, Lassa fever virus, vaccinia virus, rotavirus, and African swine virus) bind to molecular motor proteins (e.g., kinesin, dynein, and myosin), IMMP co-factors, or subunits thereof, which then deliver the viral particle to the microtubule organization center, nucleus, or other site within the cell during viral infection.

Exemplary IMMPs include kinesin, dynein (including, e.g., the light intermediate chain (DLIC) subunits, the intermediate chain (DIC) subunits, the Tctexl/rp3 light chain subunits, the roadblock light chain subunits, and the LC8 light chain subunits), and myosin (e.g., myosin V). Exemplary IMMP co-factors include, e.g., dynactin and dynein light chain (DLC).

### Diagnosis

An HPV infection or HPV-mediated disease may be diagnosed by examining HPV-infected tissue (e.g., tissue having an intraepithelial lesion or wart). For example, analysis of a cervical cytology sample (e.g., a Pap test) permits the identification of cells having an altered morphology indicative of HPV infection or a transforming pre-cancerous event. Additional methods of diagnosing an HPV infection or HPV-mediated disease are well known to those of skill in the art and include, for example, visual inspection, cytology, histology, HPV testing (e.g., Hybrid Capture™), colposcopy, cervicography, optical imaging, anoscopy, endocervical curettage, loss of heterozygosity (LOH) analysis, or measuring of viral load in cervical or vaginal secretions (e.g., a Pap sample). Methods for detecting and diagnosing HPV infection are described, for example, in U.S. Pat. Nos. 5,182,377; 5,346,811; 5,527,898; 6,218,104; 6,743,593; 6,936,443; and 7,316,908, and U.S. Patent Application Publication Nos. 2003/0027750; 2005/0118568; 2006/0110794; and 2007/0292841, hereby incorporated by reference.

### Antibody-Based Therapeutics

Antibodies that specifically bind to HPV L2 have a high affinity for HPV L2 and neutralize or prevent HPV L2 activity. Antibody therapeutics of the invention specifically bind to the carboxy-terminus of the HPV L2 protein and inhibit or reduce its interaction with IMMPs or IMMP cofactors. The use of such antibodies in the therapeutic methods of the invention is described herein. Examples of HPV L2 antibodies are described in U.S. Pat. Nos. 5,629,146; 5,885,770; and 5,968,522, hereby incorporated by reference. High affinity or specific binding is meant antibodies that bind to HPV L2 with a dissociation constant of e.g., less than 10⁻⁶M, more preferably less than 10⁻⁷M, 10⁻⁸M, 10⁻⁹M, 10⁻¹⁰M, 10⁻¹¹M, or 10⁻¹²M, and most preferably less than 10⁻¹³M, 10⁻¹⁴M, or 10⁻¹⁵M.

Pharmaceutical compositions, including excipients, of any antibodies of the invention are also encompassed by the present invention. Methods for the preparation and use of antibodies for therapeutic purposes are described in several patents, including U.S. Pat. Nos. 6,054,297; 5,821,337; 6,365,157; and 6,165,464, and are incorporated herein by reference.

Monoclonal and Polyclonal Antibodies

Methods for the generation of monoclonal and polyclonal antibodies are known in the art. These methods include the immunological method described by Kohler and Milstein (Nature 256: 495-497, 1975), Kohler and Milstein (Eur. J. Immunol. 6: 511-519, 1976), and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science 246: 1275-1281, 1989).

Human antibodies can also be produced using phage display libraries (Marks et al., J. Mol. Biol., 222: 581-597, 1991 and Winter et al., Annu. Rev. Immunol. 12: 433-455, 1994). The techniques of Cole et al. and Boerner et al. are also useful for the preparation of human monoclonal antibodies (Cole et al., supra; Boerner et al., J. Immunol. 147: 86-95, 1991).

Monoclonal antibodies can be isolated and purified using standard art-known methods. Antibodies can be screened using standard art-known methods (e.g., ELISA or Western blot analysis) against an HPV L2 polypeptide or fragment (e.g., a C-terminal fragment of the HPV L2 protein). Non-limiting examples of such techniques are described in Examples II and III of U.S. Pat. No. 6,365,157, herein incorporated by reference.

The antibody may be prepared in any mammal, including, e.g., mice, rats, rabbits, goats, and humans. The antibody may be a member of one of the following immunoglobulin classes: IgG, IgM, IgA, IgD, or IgE, and the subclasses thereof.

While the preferred animal for producing monoclonal antibodies is mouse, the invention is not so limited; in fact, human antibodies may be used. Such antibodies may be obtained by using human hybridomas (Cole et al., “Monoclonal Antibodies and Cancer Therapy,” Alan R. Liss Inc., p. 77-96, 1985).

Chimeric Antibodies

The invention also encompasses “chimeric” antibodies in which an animal antigen-binding variable domain is coupled to a human constant domain (see, e.g., U.S. Pat. No. 4,816,567; Morrison et al., Proc. Natl Acad. Sci. USA 81: 6851-6855, 1984; Boulianne et al., Nature 312: 643-646, 1984; and Neuberger et al., Nature 314: 268-270, 1985). Chimerized antibodies have, for example, constant regions derived substantially or exclusively from human antibody constant regions and variable regions derived substantially or exclusively from the sequence of the variable region from a mammal other than a human.

In the present invention, techniques developed for the production of chimeric antibodies by splicing the genes from a mouse antibody molecule of appropriate antigen specificity together with genes from a human antibody molecule can be used to produce antibodies of the present invention (see, e.g., Morrison et al., Proc. Natl. Acad. Sci. 81: 6851-6855, 1984; Neuberger et al., Nature 312: 604-608, 1984; and Takeda et al., Nature 314: 452-454, 1985).

DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the complementarity determining regions (CDRs) derived substantially or exclusively from the corresponding human antibody regions and DNA encoding CDRs derived substantially or exclusively from a mammal other than a human.

Suitable sources of DNA molecules that encode fragments of antibodies include cells (e.g., hybridomas) that express the full-length antibody. The fragments may be used by themselves as antibody equivalents or may be recombined into equivalents, as described above. The DNA deletions and recombinations described in this section may be carried out by known methods.

Humanized Antibodies

Humanized antibodies are chimeric immunoglobulins, immunoglobulin chains or fragments thereof (such as Fv, Fab, Fab′, F(ab′)₂, or other antigen-binding subsequences of antibodies) that contain minimal sequence derived from non-human immunoglobulin. Methods for humanizing non-human antibodies are well known in the art (see, e.g., Vaswani and Hamilton, Ann. Allergy Asthma Immunol. 81: 105-119, 1998 and Carter, Nature Reviews Cancer 1: 118-129, 2001). Generally, a humanized antibody has one or more amino acid residues introduced into it from a source that is non-human. These non-human amino acid residues are often referred to as import residues, which are typically taken from an import variable domain.

Humanization of an antibody for use in the compositions and methods of the present invention can be performed following the methods known in the art (see, e.g., Jones et al., Nature 321: 522-525, 1986; Riechmann et al., Nature 332: 323-329, 1988; and Verhoeyen et al., Science 239: 1534-1536, 1988) by substituting rodent (e.g., mouse) CDRs or other CDR sequences for the corresponding sequences of a human antibody. Accordingly, such humanized antibodies are chimeric antibodies, wherein substantially less than an intact human variable domain has been substituted by the corresponding sequence from a non-human species (see, for example, U.S. Pat. No. 4,816,567). In practice, humanized antibodies are typically human antibodies in which some CDR residues and possibly some framework residues are substituted by residues from analogous sites in, e.g., rodent (e.g., mouse) antibodies (Presta, Curr. Op. Struct. Biol. 2: 593-596, 1992).

Additional methods for the preparation of humanized antibodies can be found in U.S. Pat. Nos. 5,821,337, and 6,054,297, and Carter (supra), hereby incorporated by reference. The humanized antibody may be selected from any class of immunoglobulins, including IgM, IgG, IgD, IgA and IgE, and any isotype, including IgG₁, IgG₂, IgG₃, and IgG₄. In one embodiment, the constant domain is of the IgG₂ class. The humanized antibody may include sequences from more than one class or isotype, and selecting particular constant domains to optimize desired effector functions is within the ordinary skill in the art.

Functional Equivalents or Derivatives of Antibodies

The invention also includes functional equivalents or derivatives of the antibodies described in this specification. Functional equivalents or derivatives include polypeptides with amino acid sequences substantially identical to the amino acid sequence of the variable or hypervariable regions of the antibodies of the invention (e.g., at least 85%, 90%, 95%, or 99% identical). Functional equivalents have binding characteristics comparable to those of the antibodies, and include, for example, chimerized, humanized and single chain antibodies, antibody fragments, and antibodies, or fragments thereof, fused to a second protein, or fragment thereof. Methods of producing such functional equivalents are described, for example, in PCT Publication No. WO 93/21319; European Patent Application No. 239,400; PCT Publication No. WO 89/09622; European Patent Application No. 338,745; European Patent Application No. 332424; and U.S. Pat. No. 4,816,567; each of which is herein incorporated by reference.

Functional equivalents of antibodies also include single-chain antibody fragments, also known as single-chain antibodies (scFvs). Single-chain antibody fragments are recombinant polypeptides that typically bind antigens or receptors. These fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to ensure that the proper three-dimensional folding of the V_{L} and V_{H} domains occurs once they are linked, so as to maintain the target molecule binding-specificity of the whole antibody from which the single-chain antibody fragment is derived. Generally, the carboxy-terminus of the V_{L} or V_{H} sequence is covalently linked by a peptide linker to the amino-terminus of a complementary V_{L} and V_{H} sequence. Single-chain antibody fragments can be generated by molecular cloning, antibody phage display library, or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Functional equivalents include fragments of antibodies that have the same or comparable binding characteristics to those of the whole antibody. Such fragments may contain one or both Fab fragments or the F(ab′)₂ fragment. In one embodiment, the antibody fragments contain all six CDRs of the whole antibody, although fragments containing fewer than all of such regions, such as three, four or five CDRs, are also functional.

The functional equivalents may also be members of any one of the following immunoglobulin classes: IgG, IgM, IgA, IgD, or IgE, and the subclasses thereof. Equivalents of antibodies are prepared by methods known in the art. For example, fragments of antibodies may be prepared enzymatically from whole antibodies. Preferably, equivalents of antibodies are prepared from DNA encoding such equivalents. DNA encoding fragments of antibodies may be prepared by deleting all but the desired portion of the DNA that encodes the full-length antibody.

### Nucleic Acid-Based Therapeutics

The present invention features therapeutic nucleic acids that can be used to decrease the levels of viral proteins (e.g., HPV L2 protein) present in a cell for the treatment or inhibition of viral infections. Such therapeutic nucleic acids include, e.g., antisense nucleobase oligomers or small RNAs to downregulate expression of HPV L2 mRNA directly.

By binding to the complementary nucleic acid sequence (e.g., the sense or coding strand), antisense nucleobase oligomers are able to inhibit protein expression, presumably through the enzymatic cleavage of the RNA strand by RNAse H. Preferably, the antisense nucleobase oligomer is capable of reducing HPV L2 protein expression in a cell that is infected with HPV and expresses the L2 protein. Preferably, the decrease in L2 protein expression is at least 10% relative to cells treated with a control nucleobase oligomer, preferably 20% or greater, more preferably 40%, 50%, 60%, 70%, 80%, 90%, 95%, 99% or greater. Methods for selecting and preparing HPV L2 antisense nucleobase oligomers are well known in the art. For an example of the use of antisense nucleobase oligomers for the downregulation of VEGF expression, see, e.g., U.S. Pat. No. 6,410,322, hereby incorporated by reference. Methods for assaying levels of protein expression are also well known in the art and include, e.g., Western blotting, immunoprecipitation, and ELISA.

The present invention also features the use of RNA interference (RNAi) to inhibit expression of the HPV L2 protein. RNAi is a form of post-transcriptional gene silencing initiated by double-stranded RNA (dsRNA). Short (e.g., 15 to 35 nucleotides in length) double-stranded RNAs, known generally as “siRNAs,” “small RNAs,” or “microRNAs,” are effective at down-regulating gene expression in nematodes (Zamore et al., Cell 101: 25-33, 2000) and in mammalian tissue culture cell lines (Elbashir et al., Nature 411: 494-498, 2001). The therapeutic effectiveness of this approach in mammals was demonstrated in vivo by McCaffrey et al. (Nature 418:3 8-39, 2002). The small RNAs are, e.g., at least 15 nucleotides, preferably, 16, 17, 18, 19, 20, 21, 22, 23, 24, 25, 26, 27, 28, 29, 30, 31, 32, 33, 34, or 35 nucleotides in length and even up to 50 or 100 nucleotides in length (inclusive of all integers in between). Such small RNAs that are substantially identical to (e.g., 85%, 90%, 95%, or 99% identical in sequence) or complementary to any region of HPV L2 are included in the invention. It should be noted that longer dsRNA fragments, which are processed into small RNAs, may be used. Useful small RNAs can be identified by their ability to decrease HPV L2 protein expression levels or biological activity. Small RNAs can also include short hairpin RNAs in which both strands of an siRNA duplex are included within a single RNA molecule.

The specific requirements and modifications of small RNAs are known in the art and are described, for example, in PCT Publication No. WO 01/75164 and U.S. Application Publication Nos. 2006/0134787, 2005/0153918, 2005/0058982, 2005/0037988, and 2004/0203145, the relevant portions of which are herein incorporated by reference. In particular embodiments, siRNAs can be synthesized or generated by processing longer double-stranded RNAs, for example, in the presence of the enzyme dicer under conditions in which the dsRNA is processed to RNA molecules of about 17 to about 26 nucleotides. siRNAs can also be generated by expression of the corresponding DNA fragment (e.g., a hairpin DNA construct). Generally, the siRNA has a characteristic 2- to 3-nucleotide 3′ overhanging ends, preferably these are (2′-deoxy) thymidine or uracil. The siRNAs typically comprise a 3′ hydroxyl group. In some embodiments, single stranded siRNA or blunt-ended dsRNA is used. In order to further enhance the stability of the RNA, the 3′ overhangs are stabilized against degradation. In one embodiment, the RNA is stabilized by including purine nucleotides, such as adenosine or guanosine. Alternatively, substitution of pyrimidine nucleotides by modified analogs, e.g., substitution of uridine 2-nucleotide overhangs by (2′-deoxy) thymide, is tolerated and does not affect the efficiency of RNAi. The absence of a 2′ hydroxyl group significantly enhances the nuclease resistance of the overhang in tissue culture medium.

siRNA molecules can be obtained through a variety of protocols including, e.g., chemical synthesis or recombinant production using, e.g., a Drosophila in vitro system. They can be commercially obtained from companies such as Dharmacon Research Inc. or Xeragon Inc., or they can be synthesized using commercially-available kits such as the Silencer™ siRNA Construction Kit from Ambion (Catalog No. 1620) or HiScribe™ RNAi Transcription Kit from New England BioLabs (Catalog No. E2000S).

Alternatively, siRNA can be prepared using standard procedures for in vitro transcription of RNA and dsRNA annealing procedures, such as those described in Elbashir et al. (Genes Dev., 15: 188-200, 2001), Girard et al. (Nature, 442: 199-202, 2006), Aravin et al. (Nature, 442: 203-207, 2006), Grivna et al., (Genes Dev., 20: 1709-1714, 2006), and Lau et al. (Science, 313: 363-367, 2006). siRNAs may also obtained by, e.g., incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nucleotide RNAs and the RNAs can then be eluted from the gel slices. In addition, chromatography (e.g., size exclusion chromatography), glycerol gradient centrifugation, and affinity purification with antibodies can be used to isolate the small RNAs.

Short hairpin RNAs (shRNAs), as described in Yu et al. or Paddison et al. (Proc. Natl. Acad. Sci. USA 99: 6047-6052, 2002; Genes Dev. 16: 948-958, 2002; incorporated herein by reference) can also be used in the methods of the invention. shRNAs are designed such that both the sense and antisense strands are included within a single RNA molecule and connected by a loop of nucleotides (e.g., three or more nucleotides). shRNAs can be synthesized and purified using standard in vitro T7 transcription synthesis as described above and in Yu et al. (supra). shRNAs can also be subcloned into an expression vector that has the mouse U6 promoter sequences, which can then be transfected into cells and used for in vivo expression of the shRNA.

A variety of methods are available for the introduction (e.g., transfection) of nucleic acid molecules into mammalian cells. For example, there are several commercially-available transfection reagents useful for lipid-based transfection of siRNAs including, but not limited to, TransIT-TKOT™ (Mirus, Catalog No. MIR 2150), Transmessenger™ (Qiagen, Catalog No. 301525), Oligofectamine™ and Lipofectamine™ (Invitrogen, Catalog No. MIR 12252-011 and Catalog No. 13778-075), siPORT™ (Ambion, Catalog No. 1631), and DharmaFECT™ (Fisher Scientific, Catalog No. T-2001-01). Agents are also commercially available for electroporation-based methods for transfection of siRNA, such as siPORTer™ (Ambion, Catalog No. 1629). Microinjection techniques may also be used. The nucleic acid molecule may also be transcribed from an expression construct introduced into the cells, where the expression construct includes a coding sequence for transcribing the nucleic acid molecule operably linked to one or more transcriptional regulatory sequences. Where desired, plasmids, vectors, or viral vectors can also be used for the delivery of the nucleic acid molecule, and such vectors are known in the art. Additional methods are known in the art and are described, for example, in U.S. Patent Application Publication No. 2006/0058255.

### Small Molecules

The invention also features small molecules that serve therapeutic functions by, e.g., inhibiting the binding of the carboxy-terminal region of the HPV L2 protein to an IMMP or IMMP co-factor.

Methods of Screening Small Molecules

The invention features methods for the high throughput screening (HTS) of candidate small molecule agents for their ability to, e.g., inhibit the binding of the carboxy-terminal region of HPV L2 protein to IMMPs or IMMP co-factors. Candidate small molecules will also be screened for their ability to specifically inhibit the binding of HPV L2 protein to IMMPs or IMMP co-factors. In general, candidate small molecules must bind target sequences with a dissociation constant of less than 10⁻⁶ M for further consideration as a therapeutic agent of the invention.

HPV L2 protein of any origin (e.g., recombinant HPV L2 protein or L2 protein isolated from cells infected with HPV) can be used in HTS binding assays and methods. In general, fluorescence and luminescence based assays (e.g., ELISA or colorimetric assays) are used to measure binding affinities of candidate small molecules contacted with HPV L2 protein or a fragment thereof (e.g., a C-terminal fragment). Upon the identification of a candidate small molecule from a first screening process, the candidate small molecule can be further scrutinized for its binding affinity to HPV L2 protein and ability to inhibit or reduce the binding of HPV L2 protein to IMMPs or IMMP cofactors by means of a second, different HTS assay. This could be accomplished, for example, by contacting the promising candidate small molecule with alternate HPV L2 constructs to more precisely determine the binding affinity of the molecule. A discussion of HTS methodologies is found in Verkman, “Drug discovery in academia,” Am. J. Physiol. Cell Physiol. 286, C465-C474 (2004) and Dove, “Screening for content the evolution of high throughput,” Nat Biotechnol 21:859-864 (2003). Examples of HTS screening methods for the discovery of useful small molecule agents are found in, e.g., U.S. Pat. Nos. 7,279,286 and 7,276,346, which are incorporated by reference herein.

Candidate small molecules that have undergone HTS screening may be further modified to empirically improve HPV L2 binding affinities according to the design considerations discussed below.

Small Molecule Design

Small molecules of the invention can also be generated according to the principles of rational design. Computer modeling technology allows visualization of the three-dimensional atomic structure of a selected molecule (e.g., HPV L2 protein) and the design of new compounds that will interact with the selected molecule. The three-dimensional construct typically depends on data from x-ray crystallographic analyses or NMR imaging of the selected molecule or epitope. A computer graphics system enables prediction of how a candidate small molecule compound will bind to the target protein and allows experimental manipulation of the structures of the small molecule and target protein to perfect binding specificity. A prediction of what the molecule-protein interaction will be when small changes are made in one or both can be determined by using molecular mechanics software and computationally intensive computers. An example of a molecular modeling system described generally above includes the CHARMm and QUANTA programs (Polygen Corporation, Waltham, Mass.). CHARMm performs the energy minimization and molecular dynamics functions, while QUANTA performs the construction, graphic modeling and analysis of molecular structure. QUANTA allows interactive construction, modification, visualization, and analysis of the behavior of molecules that intact with each other. Another molecular modeling program that can be used to identify small molecules for use in the methods of the invention is DOCK (Kuntz Laboratory, UCSF).

Small Molecule Synthesis

Small molecules of the invention can be organic or inorganic compounds, and even nucleic acids. Specific binding to HPV L2 protein can be achieved by including chemical groups having the correct spatial location and charge in the small molecule. In a preferred embodiment, compounds are designed with hydrogen bond donor and acceptor sites arranged to be complementary to the targeted molecule or epitope. An agent is formed with chemical side groups ordered to yield the correct spatial arrangement of hydrogen bond acceptors and donors when the agent is in a specific conformation induced and stabilized by binding to the target molecule or epitope. Additional binding forces such as ionic bonds and van der Waals interactions can also be considered when synthesizing a small molecule of the invention. The likelihood of forming the desired conformation can be refined and/or optimized using molecular computational programs.

Organic compounds can be designed to be rigid, or to present hydrogen bonding groups on edge or plane, which can interact with complementary sites. Rebek, Science 235, 1478-1484 (1987) and Rebek et al., J. Am. Chem. Soc. 109, 2426-2431 (1987) have summarized these approaches and the mechanisms involved in binding of compounds to regions of proteins.

### Therapy

Therapy according to the methods described herein may be performed alone or in conjunction with a second anti-viral therapy and may be provided, e.g., at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the subject, the severity of the subject's infection, and the subject's response to treatment.

### Formulation and Administration of the Pharmaceutical Composition

The dosage and the timing of administration of an agent of the invention depend on various clinical factors including, e.g., the overall health of the subject and the severity of the symptoms of the viral (e.g., HPV) infection. The agent can be administered at any time (e.g., after diagnosis or detection of an HPV infection or an HPV-associated disease (e.g., using the diagnostic methods known in the art or described herein)), after exposure to HPV in subjects that have not yet been diagnosed with a viral infection but are at risk of such an infection (e.g., subjects suffering from or being treated for symptoms associated with HPV infection), or after a risk of developing an HPV infection is determined).

The agents of the present invention can be formulated and administered in a variety of ways (e.g., routes known for specific indications, including, but not limited to, topically, orally, subcutaneously, bronchial injection, intravenously, intracerebrally, intranasally, transdermally, intraperitoneally, intramuscularly, intrapulmonary, vaginally, rectally, intraarterially, intralesionally, parenterally, intraventricularly in the brain, or intraocularly). For example, the agent can be in the form of a pill, tablet, capsule, liquid, or sustained-release tablet for oral administration; a liquid for intravenous or subcutaneous administration; a polymer or other sustained-release vehicle for local administration; or an ointment, cream, gel, liquid, or patch for topical administration.

Continuous systemic infusion or periodic injection of the agent can be used to treat or prevent a viral (e.g., HPV) infection. Treatment can be continued for a period of time ranging from one day through the lifetime of the subject, more preferably 1 to 100 days, and most preferably 1 to 60 days, and most preferably until the symptoms of the viral infection are reduced or removed or until diagnostic tests demonstrate the absence of the virus in the subject's serum (e.g., blood) or cells. Dosages vary depending on the agent and the severity of the viral infection. The agent can be administered continuously by infusion, using a constant- or programmable-flow implantable pump, or by periodic injections. Sustained-release systems can also be used. Semipermeable, implantable membrane devices are also useful as a means for delivering the agent in certain circumstances. In another embodiment, the agent is administered locally, e.g., by inhalation, and treatment can be repeated periodically (e.g., one to two times per day, week, or month).

Therapeutic formulations are prepared using standard methods known in the art by mixing the active ingredient having the desired degree of purity with optional physiologically acceptable carriers, excipients, or stabilizers in the form of lyophilized formulations or aqueous solutions (see, for example, Remington's Pharmaceutical Sciences (20^{th} edition), Ed. A. Gennaro, 2000, Lippincott, Williams & Wilkins, Philadelphia, Pa.). Acceptable carriers include, e.g., saline; buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; or nonionic surfactants such as TWEEN™, PLURONICS™ or PEG.

Optionally, the formulation contains a pharmaceutically acceptable salt (e.g., sodium chloride) at physiological concentrations. The formulations of the invention may also contain a pharmaceutically acceptable preservative. Suitable preservative concentrations range from 0.1 to 2.0% v/v. Exemplary preservatives include, e.g., benzyl alcohol, phenol, m-cresol, methylparaben, and propylparaben. The formulations of the invention can include a pharmaceutically acceptable surfactant. Exemplary surfactants include Tween-20 and pluronic acid (F68). Suitable surfactant concentrations are, e.g., 0.005 to 0.02%.

The dosage of the agent will depend on other clinical factors, such as the weight and condition of the subject and the route of administration of the compound. For treating subjects, between approximately 0.1 mg/kg to 500 mg/kg body weight of the agent can be administered. A more preferable range is 1 mg/kg to 50 mg/kg body weight with the most preferable range being from 1 mg/kg to 25 mg/kg body weight. Depending upon the half-life of the agent in the subject, the agent can be administered 1, 2, 3, 4, or 5 times per day, 1, 2, 3, 4, 5, or 6 times per week, once every two weeks, once a month, or more or less frequently. The methods of the present invention provide for single as well as multiple administrations, given either simultaneously or over an extended period of time.

If antibodies are used in vivo for the treatment or prevention of a viral (e.g., HPV) infection, the antibodies of the invention can be administered to the subject in therapeutically effective amounts. In one embodiment, the antibodies are administered parenterally or intravenously by continuous infusion. The dose and dosage regimen depends upon the severity of the infection and the overall health of the subject. The amount of antibody administered is typically in the range of about 0.001 to about 10 mg/kg of subject weight, preferably about 0.01 to about 5 mg/kg of subject weight.

For parenteral administration, the antibodies are formulated in a unit dosage injectable form (e.g., solution, suspension, or emulsion) in association with a pharmaceutically acceptable parenteral vehicle. Such vehicles are inherently nontoxic, and non-therapeutic. Examples of such vehicles are, e.g., water, saline, Ringer's solution, dextrose solution, and 5% human serum albumin. Nonaqueous vehicles, such as fixed oils, ethyl oleate, or liposomes may also be used. The vehicle may contain minor amounts of additives, such as substances that enhance isotonicity and chemical stability (e.g., buffers and preservatives). The antibodies typically are formulated in such vehicles at concentrations of about 1 mg/ml to 10 mg/ml.

The dosage required depends on the choice of the route of administration; the nature of the formulation; the nature of the subject's illness; the subject's size, weight, surface area, age, and sex; other drugs being administered; and the judgment of the medical professional. Wide variations in the needed dosage are to be expected in view of the variety of agents available and the differing efficiencies of various routes of administration. For example, oral administration would be expected to require higher dosages than administration by intravenous injection. Variations in these dosage levels can be adjusted using standard empirical routines for optimization, as is well understood in the art. Administrations can be single or multiple (e.g., 2, 3, 6, 8, 10, 20, 50, 100, 150, or more administrations over a treatment period). Encapsulation of the agent in a suitable delivery vehicle (e.g., polymeric microparticles or implantable devices) may increase the efficiency of delivery, particularly for oral delivery.

Alternatively, a polynucleotide containing a nucleic acid sequence that, for example, reduces or inhibits the expression of the HPV L2 protein, and thus, reduces the availability of the HPV L2 for binding to an IMMP or IMMP co-factor, can be delivered to the appropriate cells in the subject. Expression of the coding sequence can be directed to any cell in the body of the subject. This can be achieved by, for example, the use of polymeric, biodegradable microparticle, or microcapsule delivery devices known in the art.

The nucleic acid can be introduced into the cells by any means appropriate for the vector employed. Many such methods are well known in the art (Watson et al., Recombinant DNA, Chapter 12, 2d edition, Scientific American Books, 1992). Examples of methods of gene delivery include, e.g., liposome-mediated transfection, electroporation, calcium phosphate/DEAE dextran methods, gene gun, and microinjection.

In gene therapy applications, genes are introduced into cells in order to achieve in vivo synthesis of a therapeutically effective genetic product. “Gene therapy” includes both conventional gene therapy, where a lasting effect is achieved by a single treatment, and the administration of gene therapeutic agents, which involves the one-time or repeated administration of a therapeutically effective DNA or RNA. Standard gene therapy methods typically allow for transient protein expression at the target site ranging from several hours to several weeks. Re-application of the nucleic acid can be utilized as needed to provide additional periods of reduced expression of HPV L2.

Another way to achieve uptake of the nucleic acid is using liposomes, which may be prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano et al., J. Mol. Med. 73: 479, 1995). Alternatively, tissue-specific targeting can be achieved by the use of tissue-specific transcriptional regulatory elements, which are known in the art. Delivery of “naked DNA” (i.e., without a delivery vehicle) to an intramuscular, intradermal, or subcutaneous site is another means to achieve in vivo expression.

Gene delivery using viral vectors (e.g., adenoviral, retroviral, lentiviral, or adeno-associated viral vectors) can also be used. Numerous vectors useful for this purpose are generally known and have been described (Miller, Human Gene Therapy 15: 14, 1990; Friedman, Science 244: 1275-1281, 1989; Eglitis and Anderson, BioTechniques 6: 608-614, 1988; Tolstoshev and Anderson, Current Opinion in Biotechnology 1: 55-61, 1990; Sharp, The Lancet 337: 1277-1278, 1991; Cornetta et al., Nucleic Acid Research and Molecular Biology 36: 311-322, 1987; Anderson, Science 226: 401-409, 1984; Moen, Blood Cells 17: 407-416, 1991; Miller and Rosman, Biotechniques 7: 980-990, 1989; Rosenberg et al., N. Engl. J. Med 323: 370, 1990, Groves et al., Nature, 362: 453-457, 1993; Horrelou et al., Neuron, 5: 393-402, 1990; Jiao et al., Nature 362: 450-453, 1993; Davidson et al., Nature Genetics 3: 2219-2223, 1993; Rubinson et al., Nature Genetics 33: 401-406, 2003; U.S. Pat. Nos. 6,180,613; 6,410,010; 5,399,346, all hereby incorporated by reference). These vectors include, e.g., adenoviral vectors and adeno-associated virus-derived vectors, retroviral vectors (e.g., Moloney murine leukemia virus-based vectors, spleen necrosis virus-based vectors, Friend murine leukemia-based vectors, lentivirus-based vectors (Lois et al., Science 295: 868-872, 2002; Rubinson et al., supra), papova virus-based vectors (e.g., SV40 viral vectors), herpes virus-based vectors, viral vectors that contain or display the vesicular stomatitis virus glycoprotein spike, Semliki-Forest virus-based vectors, hepadnavirus-based vectors, and baculovirus-based vectors.

In the relevant polynucleotides (e.g., expression vectors), the nucleic acid sequence is operatively linked to a promoter or enhancer-promoter combination. Short amino acid sequences can act as signals to direct proteins to specific intracellular compartments. Such signal sequences are described in detail in, e.g., U.S. Pat. No. 5,827,516, incorporated herein by reference in its entirety.

An ex vivo strategy can also be used for therapeutic applications. Ex vivo strategies involve transfecting or transducing cells obtained from the subject with a nucleic acid molecule. The transfected or transduced cells are then returned to the subject. The ex vivo methods include the steps of, e.g., harvesting cells from a subject, culturing the cells, transducing them with an expression vector, and maintaining the cells under conditions suitable for expression of the nucleic acid molecule or functional fragment. These methods are known in the art of molecular biology. The transduction step is accomplished by any standard means used for ex vivo gene therapy including, e.g., calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells may then be lethally irradiated (if desired) and injected or implanted into the patient.

Where sustained release administration of the agent (e.g., an antibody, peptide, or small molecule) is desired in a formulation with release characteristics suitable for the treatment of any disease or disorder requiring administration of the agent (e.g., infection by HPV), microencapsulation of the agent is contemplated. Microencapsulation of recombinant proteins for sustained release has been successfully performed with human growth hormone (rhGH), interferon-(rhIFN-), interleukin-2, and MN rgp120 (Johnson et al., Nat. Med. 2: 795-799, 1996; Yasuda, Biomed. Ther. 27: 1221-1223, 1993; Hora et al., Bio/Technology 8: 755-758 1990; Cleland, “Design and Production of Single Immunization Vaccines Using Polylactide Polyglycolide Microsphere Systems,” in “Vaccine Design: The Subunit and Adjuvant Approach,” Powell and Newman, Eds., Plenum Press: New York, pp. 439-462, 1995; WO 97/03692; WO 96/40072; WO 96/07399; and U.S. Pat. No. 5,654,010, hereby incorporated by reference).

The sustained-release formulations may include those developed using poly-lactic-coglycolic acid (PLGA) polymer. The degradation products of PLGA, lactic and glycolic acids, can be cleared quickly from the human body. Moreover, the degradability of this polymer can be adjusted from months to years depending on its molecular weight and composition (see, e.g., Lewis, “Controlled release of bioactive agents from lactide/glycolide polymer,” in M. ChasM and Dr. Langer (Eds.), Biodegradable Polymers as Drug Delivery Systems (Marcel Dekker: New York, pp. 1-41, 1990)).

### Kits of the Invention

The agent can be packaged alone or in combination with other therapeutic compounds as a kit. Non-limiting examples include, e.g., kits that contain, e.g., one pill, two pills, a powder (optionally, in combination with a pill or tablet), a suppository and a liquid in a vial, or two topical creams. The kit can include optional components that aid in the administration of the unit dose to patients, such as vials for reconstituting powder forms, syringes for injection, customized IV delivery systems, or inhalers. Additionally, the unit dose kit can contain instructions for preparation and administration of the agents of the invention. The kit may be manufactured as a single-use unit dose for a subject or multiple uses for a particular subject (e.g., at a constant dose or in which the individual compounds may vary in potency as therapy progresses). The kit may contain multiple doses suitable for administration to multiple subjects (“bulk packaging”). The kit components may be assembled in, e.g., cartons, blister packs, bottles, tubes, or vials.

### Additional Therapeutic Regimens

If desired, the subject may also be administered additional therapeutic regimens. For example, an additional therapeutic agent may be administered with the pharmaceutical compositions described herein at concentrations known to be effective for such therapeutic agents. Particularly useful therapeutic agents include, e.g., anti-viral agents, immunostimulatory agents, and immunization vaccines.

Additional therapeutic agents may be delivered separately or may be admixed into a single formulation together with the pharmaceutical composition. When agents are present in different pharmaceutical compositions, different routes of administration may be employed.

In some instances, the pharmaceutical composition and additional therapeutic agents are administered at least one hour, two hours, four hours, six hours, 10 hours, 12 hours, 18 hours, 24 hours, three days, seven days, fourteen days, or one month or more apart. The dosage and frequency of administration of each component can be controlled independently. The additional therapeutic agents described herein may be admixed with additional active or inert ingredients, e.g., in conventional pharmaceutically acceptable carriers. A pharmaceutical carrier can be any compatible, non-toxic substance suitable for the administration of the compositions of the present invention to a subject. Pharmaceutically acceptable carriers include, for example, water, saline, buffers and other compounds, described, for example, in the Merck Index, Merck & Co., Rahway, New Jersey. A slow-release formulation or a slow-release apparatus may be also be used for continuous administration. The additional therapeutic regimen may involve other therapies, including modification to the lifestyle of the subject being treated.

Exemplary HPV treatments that may be administered as an additional therapeutic regimen are described, for example, in U.S. Pat. Nos. 5,925,516; 6,096,869; 6,342,224; 6,380,157; 6,867,033; 6,926,897; and 7,172,870 and U.S. Patent Application Publication Nos. 2002/0182221; 2004/0109899; 2004/0152752; 2004/0214158; 2004/0225004; 2005/0037986; and 2005/0244433, hereby incorporated by reference.

Anti-Viral Agents

Anti-viral agents may be used as an additional therapeutic agent, either in combination with the vaccine or in a separate administration. Exemplary anti-viral agents are abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscarnet, fosfonet, fusion inhibitors, ganciclovir, ibacitabine, immunovir, idoxuridine, imiquimod, indinavir, inosine, integrase inhibitors, interferon type III, interferon type II, interferon type I, interferon, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine. Exemplary anti-viral agents are listed in, e.g., U.S. Pat. Nos. 6,093,550 and 6,894,033, hereby incorporated by reference.

Immunostimulatory Agents

Immunogenicity of the pharmaceutical composition may be significantly improved if the composition of the present invention is co-administered with an immunostimulatory agent or adjuvant. Exemplary immunostimulatory agents include aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM matrix, DC-Chol, DDA, cytokines, and other adjuvants and derivatives thereof.

Immunization Vaccines

In some instances, it may be desirable to combine the compositions of the present invention with compositions that induce protective responses against HPV and other viruses. For example, the compositions of the present invention can be administered simultaneously, separately, or sequentially with other immunization vaccines, such as Gardasil® (Merck & Co., Inc.; Rahway, N.J.) (see, e.g., U.S. Pat. Nos. 6,245,568; 6,251,678; and 6,358,744, hereby incorporated by reference). Additional immunization vaccines include vaccines against, e.g., influenza, malaria, tuberculosis, smallpox, measles, rubella, mumps, or any other vaccines known in the art.

### EXAMPLES

The present invention is illustrated by the following examples, which are in no way intended to be limiting of the invention.

### Example 1

### Cloning and expression of HPV proteins

A His-tagged L2 protein was produced by PCR amplification using the following primers:

Forward primer-
5′- AAAGGATCCTATGCGACACAAACGTTCTGC- 3′
Reverse primer-
5′- AAAGAATTCACTAGGCAGCCAAAGAGACATCT- 3′

The Eco restriction sites were used to clone the amplified L2 construct into a pET22B⁺ vector (Novagen) upstream of the histidine sequence at the carboxy-terminus. The L2-containing plasmid was transformed into BL15 cells (Invitrogen) and expressed in liquid culture by induction using IPTG at 37° C. The major capsid protein (L1) was produced in a similar manner. The plasmid was then transformed into BL21 expression cells (Invitrogen) and a 0.5 L liquid culture was induced with 1 μM IPTG (final concentration) at an optical density of 0.5. The cells were harvested four hours after induction. The cells were pelleted and the protein was purified using a Ni-NTA column (Qiagen).

### Example 2

### Protein-Protein Interaction Determination

To identify viral binding partners of motor proteins (e.g., dynein), we utilized surface plasmon resonance (SPR; Biacore) and immobilized the purified His-tagged L2 protein onto a chip via an anti-His antibody amine coupled to the SPR chip. Immobilization of the proteins was followed by the introduction of the dynein-dynactin protein complex. The dynein-dynactin complex was isolated based on the protocol described by Schroer et al. (Trends Cell Biol. 6: 212-215, 1996), hereby incorporated by reference. Alternatively, purified kinesin, myosin, or any other motor protein can be added, as well as the cytoplasmic extract itself. This process was repeated, immobilizing the entire HPV protein library on the SPR chip. The association of the HPV protein library with the dynactin-dynein complex was measured. The results of the screen showed a biochemical interaction between the HPV L2 protein and dynein, which suggests that the L2 minor capsid protein mediates intracellular transport of HPV towards the nucleus.

We next identified the L2 peptide sequence responsible for interacting with dynein. To map the dynein-binding region of L2, a series of truncated or point-mutated L2 protein open reading frames were utilized to show that only the C-terminal region of L2 was required for L2-dynein binding. The truncated L2 sequences were immobilized, as described above, on an SPR chip and their biochemical association with dynein was then measured. The results of this experiment demonstrated that the C-terminal region of the L2 protein starting at amino acid 424 is essential for dynein association.

### Example 3

### Generation and Administration of Anti-L2 Antibodies

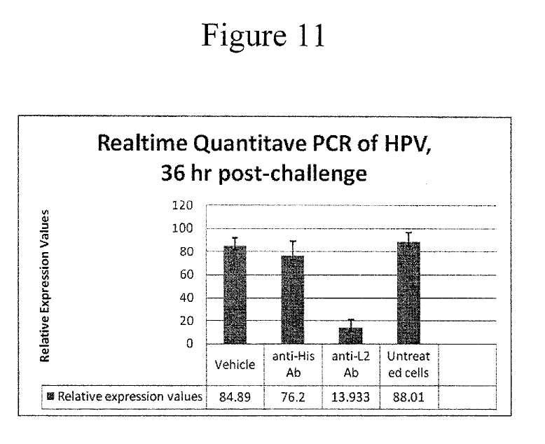

Custom antibodies were generated against the carboxy-terminal L2 sequence of PSLIPIVPGSPQYTHADGGDFYLHPSYYMLRKRRKRLPYFFSDVSLAA using the protocol described by Gönczy et al. (J. Cell Biol. 147: 135-150, 1999), hereby incorporated by reference. Alternatively, antibodies can be generated using a modified version of the in vitro immunization protocol described by Tamura et al. (Biosci. Biotechnol. Biochem. 71: 2871-2875, 2007). Using PULSin™ (Polyplus Transfection), the anti-L2 antibody targeting the dynein-interacting peptide was delivered to Vero and HeLa cells. Anti-L2 antibody was administered to the cells after forming micelles with the PULSin at amounts of 0.2 mg, 0.5 mg, and 0.75 mg for every 500,000 cells. Cells were challenged with HPV at an MOI of 1:1. Viral expression was measured by real-time PCR after 10 hours, 24 hours, and 48 hours. Control cells included untreated cells, cells transfected with only the vehicle (e.g., PULSin delivery reagent only), and cells transfected with a non-specific antibody (e.g., anti-His antibody). Results confirm that targeting the C-terminus of HPV L2 results in a significant loss of HPV expression (FIG. 11). This region can alternatively be targeted by peptide mimics or small molecules.

### Example 4

### Correlation Between Viral Protein Expression and the Presence of Dynein

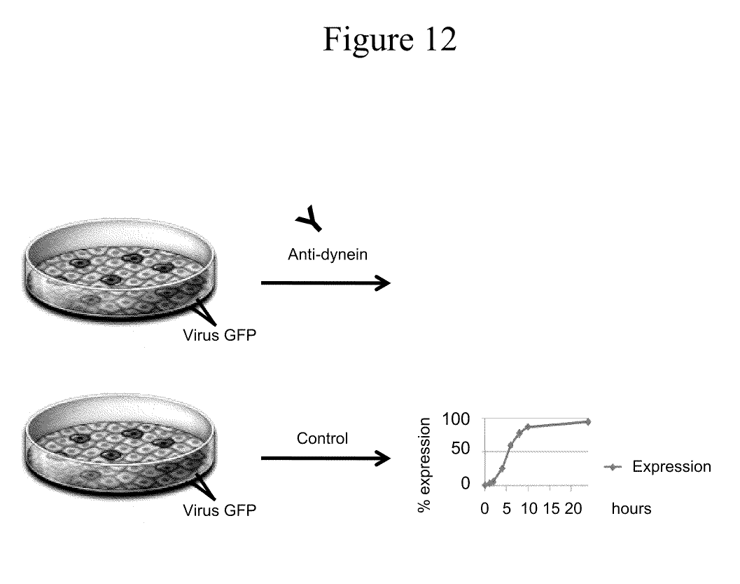

In this example, we sought to determine the function of cytoplasmic dynein in the gene expression of a variety of viruses. Three classes of viruses were chosen: herpes simplex virus (HSV-1) (a DNA virus), Moloney murine leukemia virus (MoMLV) (a retrovirus), and hepatitis C virus (Hep C) (an RNA virus). Cultured Vero cells were first treated with an anti-dynein antibody (70.1; Sigma) that targets the intermediate chain. The antibody was delivered using PULSin delivery agent (Polyplus Transfection). Anti-dynein antibody was administered to the cells after forming micelles with the PULSin reagent. Three sets of treated cells were challenged with the three classes of viruses and incubated at 37° C. Viral expression was measured by real-time PCR after 10 hours, 24 hours, and 48 hours (FIG. 12).

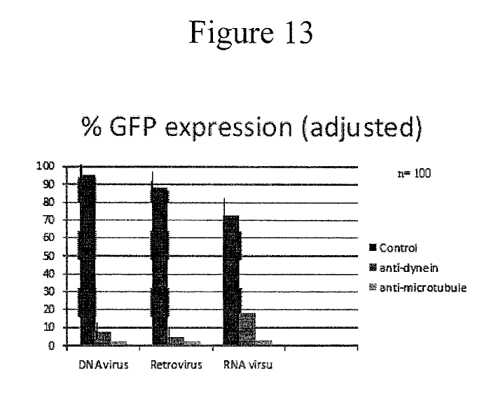

RNAi directed against dynactin was also administered to the cells in a separate experiment. Dynactin forms a complex with dynein and its cargo. Thus, we specifically silenced the expression of dynactin using RNAi. Dynactin has been shown to be required for proper cytoplasmic dynein function in several systems (see, e.g., Schroer et al., supra). To generate dsRNA corresponding to p50/dynamitin, we used the protocol described by Gönczy et al. (supra). Wild-type genomic DNA was PCR-amplified with primers corresponding to the coding sequence plus 30 nucleotides for binding of T3 (forward primer) or T7 (reverse primer) RNA polymerase (FIG. 13).

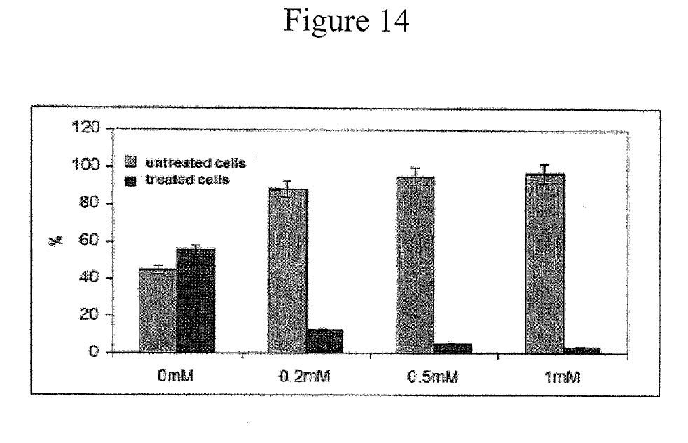

ATPase activity was inhibited using an anti-dynein small organic molecule (erythro-9-(2-hydroxy-3-nonyl)adenine; EHNA) in another set of experiments. EHNA was obtained from A. G. Scientific and dissolved in Dubelco's Modified Eagle's Medium (DMEM). The solution was added to several sets of treated cells with final concentrations of 0.1 μM, 0.2 μM, 0.5 μM, and 1 μM (FIG. 14).

In a final set of experiments, dynamitin was overexpressed in cells. Cells were transfected with p50 (dynamitin) plasmid.

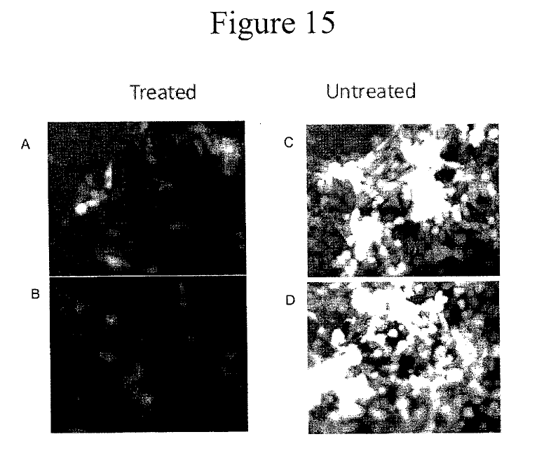

To determine the consequences of the loss of cytoplasmic dynein motor activity on the viral life cycle, we cloned the green fluorescent protein (GFP) gene into the viral genomes and assessed GFP expression upon loss of cytoplasmic dynein motor activity (FIG. 15).

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A method of inhibiting or treating infection by human papillomavirus (HPV) comprising administering a therapeutic agent that binds a carboxy-terminal region of HPV L2 protein to a subject infected with or at risk of infection by said HPV, wherein said therapeutic agent inhibits or treats said HPV infection by reducing or inhibiting the binding of said L2 protein to an intracellular molecular motor protein (IMMP) or IMMP co-factor.

2. The method of claim 1, wherein said HPV L2 protein comprises the amino acid sequence of any one of SEQ ID NOs:1-10.

3. The method of claim 1, wherein said therapeutic agent is a small molecule.

4. The method of claim 1, wherein said therapeutic agent is a peptide.

5. The method of claim 1, wherein said therapeutic agent is an antibody.

6. The method of claim 5, wherein said antibody binds a linear or conformational epitope within said carboxy-terminal region.

7. The method of claim 1, wherein said therapeutic agent is a peptidomimetic.

8. The method of claim 1, wherein said IMMP is dynein, myosin, or kinesin.

9. The method of claim 8, wherein said IMMP is dynein.

10. The method of claim 1, wherein said IMMP co-factor is dynactin.

11. The method of claim 1, wherein said carboxy-terminal region comprises the last 200 amino acid residues of said L2 protein.

12. The method of claim 1, wherein said carboxy-terminal region comprises the last 49 amino acid residues of said L2 protein.

13. The method of claim 1, wherein said carboxy-terminal region comprises the amino acid sequence: PSLIPIVPGSPQYTIIADGGDFYLHPSYYMLRKRRKRLPYFFSDVSLAA.

14. The method of claim 1, wherein said administering is by injection.

15. The method of claim 1, wherein said subject is a human.

16. The method of claim 1, further comprising administering a supplemental agent.

17. The method of claim 16, wherein said supplemental agent is an anti-viral agent, immunostimulatory agent, or immunization vaccine.

18. The method of claim 1, wherein said HPV is a strain selected from the group consisting of 1, 2, 4, 6, 7, 10, 11, 16, 18, 31, 32, 33, 35, 39, 42, 43, 44, 45, 51, 55, 56, 58, 59, and 68.

19. A method of inhibiting or treating infection by human papillomavirus (HPV) comprising administering to a subject infected with or at risk of infection by said HPV a nucleic acid molecule, wherein said nucleic acid molecule inhibits or treats said HPV infection by reducing or inhibiting expression of L2 protein in a cell infected with said HPV, wherein said reduction or inhibition of said L2 protein reduces the amount of said L2 protein available for binding to an intracellular molecular motor protein (IMMP) or IMMP co-factor, thereby inhibiting or treating said infection.

20. The method of claim 19, wherein said nucleic acid molecule is an antisense nucleic acid, a peptide nucleic acid, RNAi, shRNAi, siRNA, or micro RNAi.

21. The method of claim 19, wherein said nucleic acid molecule decreases the expression of said L2 protein in said cell by at least 50% relative to a cell not exposed to said nucleic acid molecule.

22. A pharmaceutical composition comprising an agent capable of binding the carboxy-terminal region of HPV L2 protein and reducing or inhibiting an interaction between the L2 protein and an intracellular molecular motor protein (IMMP) or IMMP co-factor.

23. The pharmaceutical composition of claim 22, wherein said HPV L2 protein comprises the amino acid sequence of any one of SEQ ID NOs:1-10.

24. The pharmaceutical composition of claim 22, wherein said IMMP is dynein, myosin, or kinesin.

25. The pharmaceutical composition of claim 24, wherein said IMMP is dynein.

26. The pharmaceutical composition of claim 22, wherein said IMMP co-factor is dynactin.

27. The pharmaceutical composition of claim 22, further comprising a pharmaceutically acceptable diluent, excipient, carrier, or adjuvant.

28. The pharmaceutical composition of claim 22, wherein said composition is suitable for administration to a human.

29. The pharmaceutical composition of claim 22, wherein said composition alleviates one or more symptoms associated with HPV.

30. The pharmaceutical composition of claim 22, wherein said agent is an antibody, peptide, or small molecule.

31. The pharmaceutical composition of claim 22, wherein said agent specifically binds to said L2 protein with a dissociation constant of less than 10⁻⁷M.

32. A pharmaceutical composition comprising a peptide corresponding to the carboxy-terminal region of the human papillomavirus (HPV) L2 protein, wherein said peptide is capable of eliciting an immune response that protects a subject against HPV infection.

33. The pharmaceutical composition of claim 32, wherein said pharmaceutical composition is formulated as a vaccine.

34. The pharmaceutical composition of claim 32, wherein said HPV L2 protein comprises the amino acid sequence of any one of SEQ ID NOs:1-10.

35. The pharmaceutical composition of claim 32, wherein said vaccine is capable of inhibiting HPV infection.

36. The pharmaceutical composition of claim 32, wherein said composition is capable of alleviating one or more symptoms associated with HPV.


SEQUENCE LISTING (10 sequences)

SEQ ID NO: 1 - protein, 473 aa, Human papillomavirus type 16
MRHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFITTPTKLITYDNPAYEGIDVDNTLYFSSNDNSINIAPDPDFLDIVALHRPALTSRRTGIRYSRIGNKQTLRTRSGKSIGAKVHYYYDFSTIDSAEEIELQTITPSTYTTTSHAALPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 2 - protein, 463 aa, Human papillomavirus type 45
MVSHRAARRKRASATDLYRTCKQSGTCPPDVINKVEGTTLADKILQWSSLGIFLGGLGIGTGSGSGGRTGYVPLGGRSNTVVDVGPTRPPVVIEPVGPTDPSIVTLVEDSSVVASGAPVPTFTGTSGFEITSSGTTTPAVLDITPTVDSVSISSTSFTNPAFSDPSIIEVPQTGEVSGNIFVGTPTSGSHGYEEIPLQTFASSGSGTEPISSTPLPTVRRVAGPRLYSRANQQVRVSTSQFLTHPSSLVTFDNPAYEPLDTTLSFEPTSNVPDSDFMDIIRLHRPALSSRRGTVRFSRLGQRATMFTRSGKQIGGRVHFYHDISPIAATEEIELQPLISATNDSDLFDVYADFPPPASTTPSTIHKSFTYPKYSLTMPSTAASSYSNVTVPLTSAWDVPIYTGPDIILPSHTPMWPSTSPTNASTTTYIGIHGTQYYLWPWYYYFPKKRKRIPYFFADGFVAA

SEQ ID NO: 3 - protein, 468 aa, Human papillomavirus type 2
MSIRAKRRKRASPTDLYRTCKQAGTCPPDIIPRVEQNTLADKLLKWGSLGVFFGGLGIGTGSGTGGRTGYIPVGSRPTTVVDIGPTPRPPVIIEPVGASEPSIVTLVEDSSIINAGASHPTFTGTGGFEVTTSTVTDPAVLDITPSGTSVQVSSSSFLNPLYTEPAIVEAPQTGEVSGHVLVSTATSGSHGYEEIPMQTFATSGGSGTEPISSTPLPGVRRVAGPRLYSRANQQVQVRDPAFLARPADLVTFDNPVYDPEETIIFQHPDLHEPPDPDFLDIVALHRPALTSRRGTVRFSRLGRRATLRTRSGKQIGARVHFYHDISPIGTEELEMEPLLPPASTDNTDMLYDVYADSDVLQPLLDELPAAPRGSLSLADTAVSATSASTLRGSTTVPLSSGIDVPVYTGPDIEPPNVPGMGPLIPVAPSLPSSVYIFGGDYYLMPSYVLWPKRRKRVHYFFADGFVAA

SEQ ID NO: 4 - protein, 463 aa, Human papillomavirus type 13
MAHSRARRRKRASATQLYQTCKASGTCPPDVIPKVEQNTLADKILKWGSLGVFFGGLGIGTGSGTGGRTGYVPVGSTPRPAISSGPTARPPIVVDTVGPTDPSIVSLVEESAIINSGVPDPLPPVHGGFEITTSQSATPAILDVSVTTQNTTSTSIFRNPVFSEPSITQSQPSIESGAHVFISPSTISPHSTEDIPLDTFIVSSSDSNPASSTPVPATVARPRLGLYSRALHQVQVTDPAFFSSPQRLITFDNPTYEGEDISLQFAHNTIHEPPDEAFMDIIRLHRPAITSRRGLVRFSRIGQRGSMYTRSGKHIGGRVHFFKDISPISAAAEQIELHPLVAAAQDHSGLFDIYAEPDPDPVAVNTSGSLSSASTPFAQSSLSSAPWGNTTVPLSLPGDIFIQPGPDITFPTAPTVTPYNPVTPILPTGPVFITASGFYLYPTWYFTRKRRKRVSLFFTDVAA

SEQ ID NO: 5 - protein, 462 aa, Human papillomavirus type 18
MVSHRAARRKRASVTDLYKTCKQSGTCPSDVVNKVEGTTLADKILQWSSLGIFLGGLGIGTGSGTGGRTGYIPLGGRSNTVVDVGPTRPPVVIEPVGPTDPSIVTLIEDSSVVTSGAPRPTFTGTSGFDITSAGTTTPAVLDITPSSTSVSISTTNFTNPAFSDPSIIEVPQTGEVSGNVFVGTPTSGTHGYEEIPLQTFASSGTGEEPISSTPLPTVRRVTGPRLYSRAYQQVSVANPEFLTRPSSLITYDNPAFEPMDTTLTFEPRSNVPDSDFMDIIRLHRPASTSRRGTVRFSRLGQRATMFTRSGTQIGARVHFYHDISPIAPSPEYIELQPLVSATEDNGLFDIYADDIDPALPVPSRPTTSSAVSTYSPTISSASSYSNVTVPLTSSWDVPVYTGPDITLPSTTSVWPIVSPTDPASTQYIGIHGTHYYLWPLYYFIPKKRKRVPYFFADGFVAA

SEQ ID NO: 6 - protein, 464 aa, Human papillomavirus type 56
MVAHRATRRKRASATQLYKTCKLSGTCPEDVVNKIEQKTWADRILQWGSLFTYFGGLGIGTGTGSGGRAGYVPLGSRPSTIVDVTPARPPIVVESVGPTDPSIVTLVEESSVIESGAGIPNFTGSGGFEITSSSTTTPAVLDITPTSSTVHVSSTHITNPLFIDPPVIEAPQTGEVSGNILISTPTSGIHSYEEIPMQTFAVHGSGTEPISSTPIPGFRRIAAPRLYRKAFQQVKVTDPAFLDRPATLVSADNPLFEGTDTSLAFSPSGVAPDPDFMNIVALHRPAFTTRRGGVRFSRLGRKATIQTRRGTQIGARVHYYYDISPIAQAEEIEMQPLLSANNSFDGLYDIYANIDDEAPGLSSQSVATPSAHLPIKPSTLSFASNTTNVTAPLGNVWEAPFYSGPDIVLPTGPSTWPFVPQSPYDVTHDVYIQGSSFALWPVYFFRRRRRKRIPYFFADGDVAA

SEQ ID NO: 7 - protein, 476 aa, Human papillomavirus type 32
MPPHRSRRRKRASATQLYQTCKASGTCPPDVIPKIEGRTWADQILKWGSTGVFFGGLGIGTGAGSGGRTGYVPIGTRPPVVAEPGPAIRPPVVVDTIGPTDPSVISLLEESAVIDSSIPVPTDTSHGGFNITSSASGPSSTPAVLDISPPTNTIRVASTTSHNPVYSDPFTLRPSLPVEGNGRLLTSHPTIAPHSYEEIPMDTFVVSTDTSNTVTSTPIPGPRPTMRLGLYTRVTQQRPVATTTFLTSPERLVTYDNPAYEGPAEGTLEFEHPTIHEAPDSDFMDIIALHRPVLSARQGTVRVSRIGQRASLQTRSGARIGSRVHFFHDISPITRPSEAIELQPLGSSSTAVSTTASSAINDGLFDVYVDPDIPPSHALPPLRSPTHVSTVSLTSLGSVPAQTANTTVPLSLPTNINVGPDLSPPESPPFISTRPVSPSFDSVMVLGWDFILHPSYMWRKRRKPVPYFFADVRVAA

SEQ ID NO: 8 - protein, 466 aa, Human papillomavirus type 31
MRSKRSTKRTKRASATQLYQTCKAAGTCPSDVIPKIEHTTIADQILRYGSMGVFFGGLGIGSGSGTGGRTGYVPLSTRPSTVSEASIPIRPPVSIDPVGPLDPSIVSLVEESGIVDVGAPAPIPHPPTTSGFDIATTADTTPAILDVTSVSTHENPTFTDPSVLQPPTPAETSGHLLLSSSSISTHNYEEIPMDTFIVSTNNENITSSTPIPGVRRPARLGLYSKATQQVKVIDPTFLSAPKQLITYENPAYETVNAEESLYFSNTSHNIAPDPDFLDIIALHRPALTSRRNTVRYSRLGNKQTLRTRSGATIGARVHYYYDISSINPAGESIEMQPLGASATTTSTLNDGLYDIYADTDFTVDTPATHNVSPSTAVQSTSAVSAYVPTNTTVPLSTGFDIPIFSGPDVPIEHAPTQVFPFPLAPTTPQVSIFVDGGDFYLHPSYYMLKRRRKRVSYFFTDVSVAA

SEQ ID NO: 9 - protein, 467 aa, Human papillomavirus type 33
MRHKRSTRRKRASATQLYQTCKATGTCPPDVIPKVEGSTIADQILKYGSLGVFFGGLGIGTGSGSGGRTGYVPIGTDPPTAAIPLQPIRPPVTVDTVGPLDSSIVSLIEETSFIEAGAPAPSIPTPSGFDVTTSADTTPAIINVSSVGESSIQTISTHLNPTFTEPSVLHPPAPAEASGHFIFSSPTVSTQSYENIPMDTFVVSTDSSNVTSSTPIPGSRPVARLGLYSRNTQQVKVVDPAFLTSPHKLITYDNPAFESFDPEDTLQFQHSDISPAPDPDFLDIIALHRPAITSRRHTVRFSRVGQKATLKTRSGKQIGARIHYYQDLSPIVPLDHTVPNEQYELQPLHDTSTSSYSINDGLYDVYADDVDNVHTPMQHSYSTFATTRTSNVSIPLNTGFDTPVMSGPDIPSPLFPTSSPFVPISPFFPFDTIVVDGADFVLHPSYFILRRRRKRFPYFFTDVRVAA

SEQ ID NO: 10 - protein, 469 aa, Human papillomavirus type 35
MRHKRSTKRVKRASATQLYRTCKAAGTCPPDVIPKVEGNTVADQILKYGSMAVFFGGLGIGSGSGTGGRSGYVPLGTTPPTAATNIPIRPPVTVESIPLDTIGPLDSSIVSLVEETSFIESGAPVVTPRVPPTTGFTITTSTDTTPAILDVTSISTHDNPTFTDPSVLHPPTPAETSGHFVLSSSSISTHNYEEIPMDTFIVSTDSNNITNSTPIPGSRPTTRLGLYSKGTQQVKVVDPAFMTSPAKLITYDNPAYEGLNPDTTLQFEHEDISLAPDPDFMDIIALHRPALTSRKGTIRYSRVGNKRTMHTRSGKAIGARVHYYQDLSSITEDIELQPLQHVPSSLPHTTVSTSLNDGMFDIYAPIDTEEDIIFSASSNNTLYTTSNTAYVPSNTTIPLSSGYDIPITAGPDIVFNSNTITNSVLPVPTGPIYSIIADGGDFYLHPSYYLLKRRRKAIPYFFADVSVAV